(19) United States
(12) Patent Application Publication
(10) Pub. No.: US 2026/0266802 A1
(43) Pub. Date: Sep. 10, 2026

(54) SYSTEMS AND METHODS FOR REAL-TIME DETECTION AND CAPTURE OF INVASIVE CELL SUBPOPULATIONS FROM CO-CULTURES

(71) Applicant: Georgetown University, Washington, DC (US)

(72) Inventors: Anton Wellstein, Washington, DC (US); Ghada M. Sharif, Arlington, VA (US); Makarand Paranjape, Ashton, MD (US); Leon Der, New Carrolton, MD (US)

(21) Appl. No.: 18/849,375

(22) PCT Filed: Mar. 24, 2023
(86) PCT No.: PCT/US2023/064932

§ 371 Date: Sep. 20, 2024

Related U.S. Application Data
(60) Provisional application No. 63/323,418, filed on Mar. 24, 2022.
(60) Provisional application No. 63/355,335, filed on Jun. 24, 2022.

(51) Int. Cl. G01N 33/50; G01N 15/01; G01N 15/10; G01N 15/1031; G01N 33/483

(52) U.S. Cl. CPC G01N 33/5005; G01N 15/01; G01N 15/1031; G01N 33/4833; G01N 2015/1006

(57) ABSTRACT

Systems and methods are disclosed for identifying invasive cells. In certain embodiments, the system is comprised of a first chamber comprised of media, and the first plurality of cells are healthy cells, diseased cells, or a mixture of both, a second chamber comprised of a filtration membrane and a third chamber comprised of media, and a second plurality of cells. The second plurality of cells produces one or more chemoattractants or chemorepellents, and the first, second, and third chambers are clipped together to form a chamber array. The chamber array is mounted to an electrical cell impedance sensing reader, and impedance readouts are collected from the electrical cell impedance sensing reader at predetermined time intervals. Then, invasive cells are extracted from under the filtration membrane of the second chamber, wherein the extracted invasive cells are characterized using the collected impedance readouts normalized over a predetermined time period.

### CROSS-REFERENCE TO RELATED APPLICATIONS

This application claims the benefit of U.S. Provisional App. No. 63/323,418, filed Mar. 24, 2022, and U.S. Provisional App. No. 63/355,335, filed Jun. 24, 2022, the entire contents of which are both incorporated herein in their entireties.

### GOVERNMENT LICENSE RIGHTS

This invention was made with government support under Grant Nos. R01CA205632 and R21CA226542, awarded by the National Institute of Health. The government has certain rights in the invention.

### FIELD OF THE INVENTION

The present invention relates to the field of cell-based assays. In particular, the invention provides impedance-based devices, apparatuses and systems for analyzing cells and for conducting cell-based assays.

### BACKGROUND OF THE INVENTION

Bioelectronics is a progressing interdisciplinary research field that involves the integration of biomaterials with electronic devices. Bioelectronic methods have been used for analyzing cells and assaying biological molecules and cells. In one type of application, cells are cultured on microelectrodes and cell-electrode impedance is measured and determined to monitor cellular changes.

In PCT Application No. PCT/US03/22557, entitled “IMPEDANCE BASED DEVICES AND METHODS FOR USE IN ASSAYS”, filed on Jul. 18, 2003, a device for detecting cells and/or molecules on an electrode surface is disclosed. The device detects cells and/or molecules through measurement of impedance changes resulting from the attachment or binding of cells and/or molecules to the electrode surfaces. A number of embodiments of the device are disclosed, together with the apparatuses and system for using such devices to perform certain cell-based assays.

The study of time dependence of cytotoxic and cell proliferation inhibitory effect of a drug is an important element for gaining information to use in the development of clinical dosing strategies. In particular, time dependent IC50's are derived and different time dependent patterns for IC50's are observed (e.g., see Hassan S B, Jonsson E, Larsson R and Karlsson M O in J. Pharmacology and Experimental Therapeutics, 2001, Vol. 299, No. 3, pp 1140-1147; Levasseur L M, Slocum H K, Rustum Y M and Greco W R, in Cancer Research, 1998, vol. 58, pp 5749-5761.). Typically, these studies used end-point single-measurement assays. Each time point for a dose concentration of drug or compound applied to the cultured cells required a separate experiment. That limits the time resolution and the number of time points of such time-dependent cytotoxicity studies. Thus, new technologies or methods that can provide higher time resolution and permit measurements on many time points are needed.

### SUMMARY OF THE INVENTION

Described herein are systems and methods for identifying invasive cells. In certain embodiments, the system is comprised of a first chamber comprised of media, and the first plurality of cells are healthy cells, diseased cells, or a mixture of both, a second chamber comprised of a filtration membrane and a third chamber comprised of media, and a second plurality of cells. The second plurality of cells produces one or more chemoattractants or chemorepellents, and the first, second, and third chambers are clipped together to form a chamber array. The chamber array is mounted to an electrical cell impedance sensing reader, and impedance readouts are collected from the electrical cell impedance sensing reader at predetermined time intervals. Then, invasive cells are extracted from under the filtration membrane of the second chamber, wherein the extracted invasive cells are characterized using the collected impedance readouts normalized over a predetermined time period.

In certain embodiments, the invasive cells are immune cells, epithelial cells, neuronal cells, glial cells, fibroblasts, or endothelial cells.

In other embodiments, the chamber array is formed by cutting and interlocking mechanical protrusions in the first, second, and third chambers.

In yet other embodiments, the media used in the system is serum-free.

In other embodiments, the filtration membrane is comprised of polyethersulfone.

### BRIEF DESCRIPTION OF THE DRAWINGS

A more complete appreciation of the invention and many of the attendant advantages thereof will be readily obtained as the same becomes better understood by reference to the following detailed description when considered in connection with the accompanying drawings, wherein:

FIG. 1A shows a diagram outlining the process followed by a cell invasion monitoring and cell collection system, in accordance with an exemplary embodiment of the invention;

FIG. 1B shows a diagram of the cell invasion monitoring and cell collection system as assembled;

FIG. 1C shows a diagram of the cell invasion monitoring and cell collection system during collection of cells;

FIG. 2A is an image of the three chambers used to build the cell invasion monitoring and cell collection system;

FIG. 2B is an image of the middle chamber of the cell invasion monitoring and cell collection system;

FIG. 2C is an image of the lower chamber of the cell invasion monitoring and cell collection system;

FIG. 3A is a chart showing real-time cellular analysis of MDA-MD-231 cell invasion, alone or in co-culture with 3T3-J2 fibroblasts (bottom chamber);

FIG. 3B is a chart showing real-time cellular analysis (RTCA) of DCIS-44 cell invasion, alone or in co-culture with 3T3-J2 fibroblasts (bottom chamber);

FIG. 4 is a chart showing real-time cellular analysis (RTCA) of Human Umbilical Vein Endothelial Cells (HUVEC) invasion, alone, in co-culture with the invasive MDA-MB-231 cells (bottom chamber) or the non-invasive Ductal Carcinoma In Situ (DCIS) cells (bottom chamber);

FIG. 5 is a chart showing cell invasion of patient derived xenografts (PDX) co-cultured with bone marrow immune cells;

FIG. 6A is a diagram of the cell invasion monitoring and cell collection system comprising migrating MDA-MB-231 cells, in which HUVEC cells in the system are treated with palbociclib; and

FIG. 6B is a chart showing the migration of MDA-MB-231 cells in a system where HUVEC cells in the system are treated with palbociclib, as monitored in real-time.

### DETAILED DESCRIPTION OF THE PREFERRED EMBODIMENTS

In describing a preferred embodiment of the invention illustrated in the drawings, specific terminology will be resorted to for the sake of clarity. However, the invention is not intended to be limited to the specific terms so selected, and it is to be understood that each specific term includes all technical equivalents that operate in a similar manner to accomplish a similar purpose. Several preferred embodiments of the invention are described for illustrative purposes, it being understood that the invention may be embodied in other forms not specifically shown in the drawings.

Invasion and metastatic spread of cancer cells are the major cause of death from cancer. Assays developed early on to measure the invasive potential of cancer cell populations typically generate a single endpoint measurement that does not distinguish between cancer cell subpopulations with different invasive potential. Also, the tumor microenvironment consists of different resident stromal and immune cells that alter and participate in the invasive behavior of cancer cells. Invasion into tissues also plays a role for immune cell subpopulations fending off microorganisms or eliminating diseased cells from the parenchyma as well as endothelial cells during tissue remodeling and angiogenesis. Real-Time Cellular Analysis (RTCA) that utilizes impedance biosensors to monitor cell invasion was a major step forward beyond endpoint measurement of invasion: RTCA provides continuous measurements over time and thus can reveal differences in invasion rates that are lost in the endpoint assay. Using current RTCA technology, we expanded dual-chamber arrays by adding a further chamber that can contain stromal and/or immune cells and allows measuring the rate of invasion under the influence of secreted factors from co-cultured stromal or immune cells over time. Beyond that, the unique design allows for detaching chambers at any time and isolating of the most invasive cancer cell or other cell subpopulations that are present in heterogeneous mixes of tumor isolates tested.

Those most invasive cancer cell and other cell subpopulations drive malignant progression to metastatic disease and their molecular characteristics are important for in-depth mechanistic studies, the development of diagnostic probes for their detection and assessment of vulnerabilities. Thus, inclusion of small-or large-molecule drugs can be used to test the potential of therapies that target cancer and/or stromal cell subpopulations with the goal of inhibiting (e.g. cancer cells) or enhancing (e.g. immune cells) invasive behavior.

Cell invasion is an important process that allows cells to cross basement membrane barriers in response to environmental cues provided by stromal cells. It is a crucial step during several stages of development, for immune responses, wound healing, tissue repair and in malignancies that can progress from local lesions to invasive and metastatic cancers. Assays developed early on to measure the invasive potential of cell populations typically generate a single endpoint measurement or require pre-labeling of invasive cells. The integration of microelectronics and microfluidics techniques are now developed to detect different aspects of cell biology such as viability, movement and attachment using electric impedance of live cells on microelectrodes. Impedance measurement allows for a label free, non-invasive and quantitative assessment of cell status. Described herein is a three chambered array based on the design of the Real-Time Cellular Analysis (RTCA) system that was developed by Abassi et al. The three chambered array allows for the assessment of co-cultured cells on cellular invasion and recovery of invasive cells for additional analyses or expansion.

In the RTCA system, cells invade through an extracellular matrix that is coated onto a porous membrane and reach an interdigitated electrode array that is positioned on the opposite side of the barrier. As the invasive cells continue to attach and occupy that electrode array over time, the electrical impedance changes in parallel. The current system is comprised of a cell invasion and migration (CIM) 16 well plate with two chambers. The RTCA-DP (dual purpose) instrument contains sensors for impedance measurement and integrated software to analyze and process the impedance data. Impedance values at baseline depend on the ionic strength of media in the wells and are changed as cells attach to the electrodes. The impedance changes depend on the number of cells, their morphology and the extent to which cells attach to the electrodes. A measurement of wells with media before cells are added is considered the background signal.

Background is subtracted from impedance measurements after reaching equilibrium with cells attaching and spreading onto the electrodes. A unitless parameter of the status of the cells on an electrode termed Cell Index (CI) is calculated as follows: CI=(impedance after equilibrium minus impedance in the absence of cells)/nominal impedance value. When migration rates of different cell lines are compared, the Delta CI can be used to compare cell status regardless of the difference in attachment that is represented in the first few measurements.

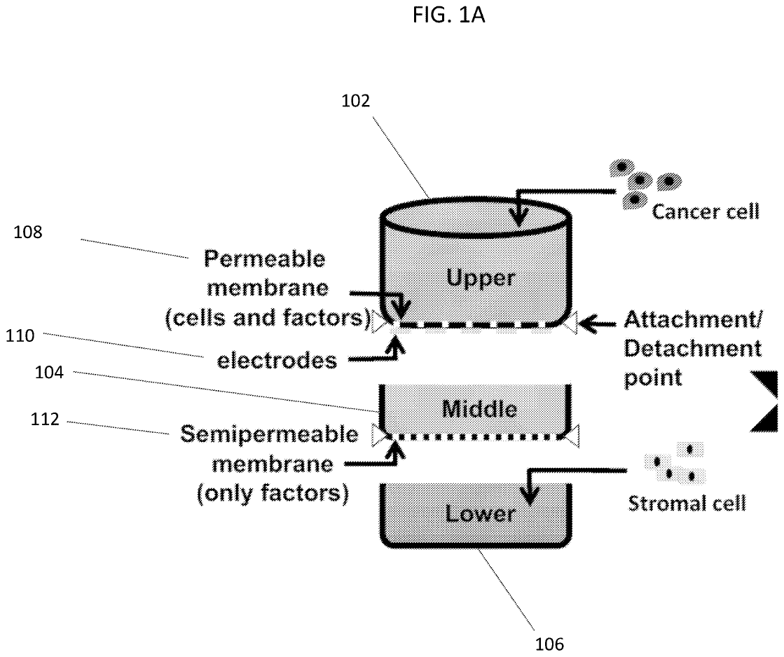

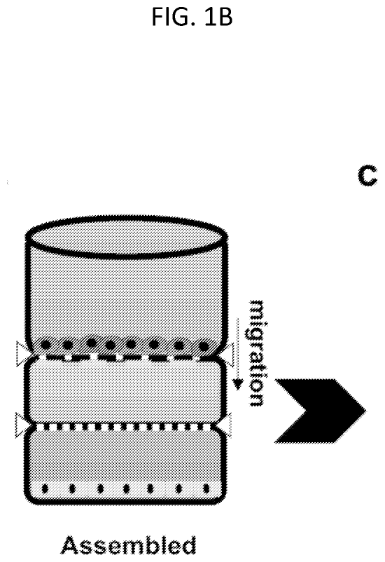

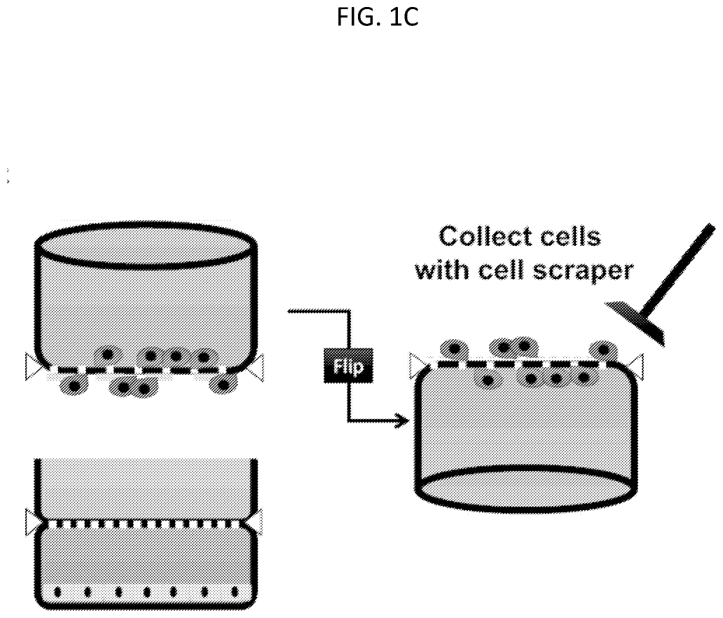

The newly designed three-chambered array builds on the existing design and uses the top chamber from the RTCA-DP system that contains the electrodes. The modified middle and bottom chambers are adapted to fit the assembly into the RTCA-DP instrument for impedance measurement and analysis using the integrated software. Two major advances the new design provides over the existing dual chamber CIM-plate are i) the ability to recover and then analyze invasive cell subpopulations that are present in heterogeneous cell mixes and ii) the option to assess the impact of secreted factors from co-cultured stromal or immune cells on cell invasion, as shown with regard to FIGS. 1A to 1C.

FIG. 1A is a diagram outlining the process followed by a cell invasion monitoring and cell collection system, in accordance with an exemplary embodiment of the invention. In certain embodiments, the system is comprised of an upper chamber 102, a middle chamber 104, and a lower chamber 106. The upper chamber 102 has impedance biosensors and receives the cell line to be monitored. The upper chamber 102 and the middle chamber are separated by a permeable membrane 108. The permeable membrane 108 allows for the passage of cells and factors between the upper chamber 102 and the middle chamber 104. A plurality of electrodes 110 are also situated between the upper chamber 102. The electrodes 110 assist in cell-electrode impedance measurements that can be used to determine and monitor cellular changes. The middle chamber 104 and the lower chamber 106 are separated by a semipermeable membrane 112 that selectively allows the passage of cell factors, but not the cells themselves. Stromal and/or immune cells may be found in the lower chamber 106. Those cells in the lower chamber 106 may produce one or more chemoattractants or chemorepellents.

The system of the present invention is assembled as shown in FIG. 1B. Cells are added to the upper chamber 102. Real-time cell invasion/migration from the upper chamber 102 towards the lower chamber 106 is recorded until a user-defined timepoint for cell collection is reached. The middle chamber 104 and bottom chamber 106 are modified and adapted to fit the assembly into a RTCA-DP instrument for impedance measurement and analysis using integrated software. As cells move towards the bottom chamber, it is possible to recover and then analyze invasive cell subpopulations that are present in heterogeneous cell mixes and assess the impact of secreted factors from co-cultured stromal or immune cells on cell invasion.

FIG. 1C shows extraction of invasive cells from the heterogeneous cell mixture. Upon completion of the process described, the upper chamber 102 is dismantled and inverted. Then, cells are harvested using a cell lifter.

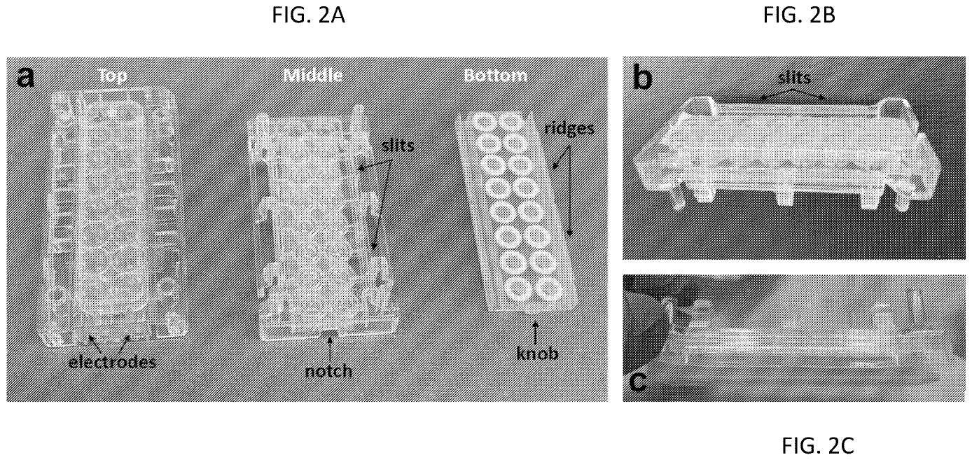

FIGS. 2A through 2C show images of the array chambers and modifications. FIG. 2A shows the three chambers used to build the array. No modification was performed on the top chamber 102 harboring the electrodes 110. With regard to the middle chamber 104, it is preferably modified such that approximately 2 mm are shaved off the middle chamber 104 wells and a membrane 108 is attached to the open bottom. Additionally, longitudinal slits, preferably 1.5 mm×5.6 mm, are added to each side. The lower chamber 106, preferably 72 mm×18 mm, is fabricated to replicate the 16-well design, and wells are preferably 4.8 mm deep and 4.75 mm in diameter, with triangle ridges, preferably 1.5 mm horizontal×1.4 mm vertical, added along the sides to click into the middle chamber 104 slits. One of ordinary skill will readily understand that the modifications to the middle chamber 104 and the lower chamber 106 described herein may be varied based on the requirements and circumstances of the analysis being performed. Example 1, as described below, articulates the process exemplarily followed in order to analyze invasive cells using the system described with regard to FIGS. 1A-1C and 2A-2C.

### Example 1: Process for Analyzing Invasive Cells

1. New chamber design
1.1 Open a new dual chamber CIM-plate. Set aside the top chamber with electrodes.
1.2 Using a milling machine, shave off 2 mm of the u-shaped bottom wells of the CIM-plate.
1.3 Attach a 2×7 cm polyethersulfone (PES) membrane with 0.2 μm pore size to the bottom of the shaved wells using UV-curated adhesive. Allow 30 minutes curation time to ensure glue is completely cured and inert.
1.4 Using a milling machine, cut out two longitudinal slits (1.5 mm×5.6 mm) along the sides to snap into the ridges of the newly fabricated third chamber.
1.5 Using a milling machine, create a polycarbonate third chamber that replicates the overall dimensions of the RTCA CIM-plate; 72 mm×18 mm.
1.6 Create wells 4.8 mm deep and 4.75 mm in diameter to replicate the 16 well design of the RTCA CIM-plate. That allows 90ul of volume per well.
1.7 On the sides, create two triangular ridges so that the chamber locks into the original's slits created in step 1.3. The horizontal part of the triangle is 1.5 mm; the vertical is 1.4 mm and the hypotenuse is 2.052 mm.
1.8 Create a knob on the short side that is 50.8 mm in diameter and 1.397 mm height to fit into the original's notch.
1.9 Use a 0.9 mm thick rubber washer for each well to provide a sealed fit.

### 2. Cell Culture (mda-mb-231, Dcis, Dcis-44, J2-fibroblasts)

2.1 Wash adherent cell cultures of ~70% confluence with 1× phosphate buffered saline (PBS).
2.2 Add 0.05% trypsin-EDTA solution to lift cells off.
2.3 Neutralize the trypsin solution with cell culture media containing serum and count the cells using an aliquot of the cell suspension.

Note: The specific cell culture media can be found in the Materials Table.

### 3. Patient Derived Xenograft Dissociation

3.1 Chop a fresh tumor piece (1 cm²) into fine mush using a sterile scalpel.
3.2 Place in a 50 ml conical tube with 20 mL DMEM F12 media supplemented with 3mg/mL trypsin and 2 mg/mL collagenase.
3.3 Incubate in a thermal shaker (150 RPM) at 37° C. for 20 min.
3.4 Spin tube at 500 g for 5 min, remove supernatant.
3.5 Wash 3 times with 20 ml of DMEM F12+2% FBS.
3.6 Resuspend in PDX media (see Materials table) to count cell suspension.

### 4. Bone Marrow Cell Extraction

Note: The study was reviewed and considered as “exempt” by the Institutional Review Board of Georgetown University (IRB #2002-022). Freshly harvested bone marrow tissues were collected from discarded healthy human bone marrow collection filters that had been de-identified.
4.1 Flush bone marrow (BM) collection filter with 25 mL of 1× PBS.
4.2 Add the flushed BM slowly to a 50 ml conical tube with 25 mL of Lymphoprep, taking care to keep the layers as separate as possible.
4.3 Spin at 800 g for 20 mins at 18° C.
4.4 Siphon off the top layers after centrifugation (fat/plasma) and transfer the 5 mL white layer above the Lymphoprep that has the BM cells to a 15 ml conical tube. Alternatively, you can dip a 5 mL pipette into the top layer until it touches the middle layer (BM), slurp up the middle layer very slowly without moving the pipette.
4.5 Fill the 15 ml conical tube with 1× PBS (~10 ml) and spin at 300 g for 15 mins.
4.6 Remove the supernatant, the remaining white pellet is the BM.
4.7 If red blood cells are observed in the pellet, add 5 mL RBC lysis solution and let sit for 5 mins at room temperature.
4.8 Wash with 1XPBS. Repeat until the pellet is white.

### 5. Cell Seeding and Assembly

5.1 Place all three sterile chambers in the tissue culture hood.
5.2 Locate the knob on the short side of the lower chamber, orient the lower chamber so that the knob is facing you.
5.3 Add 30,000 to 50,000 cells in 90ul media to each well of the lower chamber. Avoid forming bubbles. These are the stromal cells that will provide secreted factor but will not be detected by the electrodes of the top chamber.
5.3.1 Controls: Use 5% fetal bovine serum supplemented media in two of the lower chamber wells as a positive control for cell motility. Use 0% serum supplemented media as a negative control.
5.4 Let the lower chamber with the cells sit for 10-15 minutes in the hood for cells to settle. Note: this step is recommended if cells are adherent or grow in suspension.
5.5 Rotate the lower chamber 90 degrees and place the middle chamber on top of it so that the knob on the lower chamber slides into the notch on the middle chamber.

Note: The knob on the lower chamber and the blue dot on the middle chamber are at opposite ends of the assembly.
5.6 Push vertically down until you hear a click sound from each of the long sides of the assembly.
5.7 Add 160 μl serum free media to all wells of the middle chamber.
5.7.1 Make sure a dome shaped meniscus is visible after wells are filled, otherwise adjust the final volume based on your pipette calibration. Avoid forming bubbles.
5.8 Place the top chamber with electrodes facing down onto the middle chamber making sure to align the blue dots on the middle and top chambers.
5.9 Push vertically down until you hear a click sound from each of the long sides of the assembly.
5.10 Add 25-50 μl of serum free media to the top chamber.
5.11 Mount the assembly on the RTCA DP analyzer in the tissue culture incubator and wait 30 minutes before measuring background.

Note: This time is necessary to equilibrate the array and can be used to prepare the cell lines to be added to the top chamber.
5.12 Measure background (see section 6) and place the assembly back into the tissue culture hood.
5.13 Add 30,000 to 50,000 cells in 100 μl serum free media to each well of the top chamber. These are the cells that will be detected by the electrode once they successfully migrate through the membrane.

Note: It is recommended to grow cells in serum free or low serum media for 6-18 hours before performing the assay to achieve maximum response.
5.14 Let stand in the hood for 30 mins before mounting on the RCTA-DP instrument for impedance measurement.

### 6. Background and Impedance Measurement

6.1 Place the array into the cradle in the RTCA DP instrument.
6.2 Open the RTCA software and select the cradle to be used.
6.3 Click on the message tab and make sure it says “connections ok” to ensure the array is well placed in the cradle and the electrodes are well aligned with the sensors.

6.4 Click on the experiment notes tab and fill as much information about your experiment as possible.
6.5 Click on the layout tab and fill in the description of your array layout.
6.6 Click on the schedule tab and add 2 steps from the steps menu; a background step (one sweep) and a test step with 100 sweeps, a sweep every 15 min, totaling 25 hrs.
6.7 After the array has been in the RTCA DP instrument incubator for 30 mins, click on the play icon to start background measurement. A window asking you to choose the folder to save the data in will pop up.
6.8 After the background measurement is done, remove the array from the cradle and back to the cell culture hood.
6.9 Add cells to the top chamber as described in step 5.13, and keep the assembly in the tissue culture hood for 30 min for cells to settle.
6.10 Place the array back into the RTCA DP instrument and check the message tab for “connections ok” message.
6.11 Click on the play icon to start impedance measurement.
6.12 Click on the plot tab to monitor progress of signal.
6.13 If the end point is reached before 25 hrs, click on abort step from the execute drop down menu.
6.14 To export data, right click on the graph and choose copy in list format then paste data in an excel sheet.

Note: you can export data as cell index or delta cell index.

Note: you can also choose to export graph and/or layout information.

### 7. Detachment and Cell Collection

7.1 Monitor the migration rate in real-time on the RTCA DP analyzer to determine the stopping point of interest (6-18 hours).

Note: the stopping point depends on the cell's invasion rate and when a distinct invasion signal from the negative control is achieved.
7.2 Once achieved, unmount the assembly from the RTCA DP analyzer and place in the tissue culture hood.
7.3 Prepare an appropriate number of 1.5 mL Eppendorf tubes to collect the cells from the wells of interest.
7.4 Place the assembly in a 10 cm dish to contain liquids when chambers detach.
7.5 Push the flexible snapping ends on either long sides of the middle chamber inwards until you hear a click.
7.6 Dismantle the top chamber and invert into a new 10 cm dish.
7.7 Use a cell lifter with 13 mm blade to collect the cells from all wells harboring the same experimental condition (i.e. cell type, drug treatment . . . etc).

Note: design your setup to have at least two wells for each experimental condition to achieve statistically significant change from negative controls.
7.8 Rinse or dip the blade in 1× phosphate buffered saline to collect cells into the 1.5 mL Eppendorf tubes.
7.9 Spin down cells at 500× g for 5min
7.10 Collected cells can be propagated (see section 8) or end point analysis can be performed such as single cell RNA-seq.

Note: for bulk RNA-seq we recommend using a low cell number RNA extraction kit.

### 8. 3d Cell Propagation and Retrieval

Due to the small number of cells collected, seed cells in 3D using extracellular matrix (ECM) to enhance viability. That said, 2D culture is also an option at this point specially if the cells used are from established cell lines.
8.1 Thaw an aliquot of Matrigel at 4C overnight. Keep Matrigel on ice until ready to use.
8.2 Add 25 ul cold Matrigel to the pellet of live cells collected and gently pipette up and down to mix. Avoid forming bubbles.
8.3 Add the cell-Matrigel mix to the bottom of a small tissue culture well (i.e. in a 96-well plate) forming a dome. Try not to touch the walls of the well.

Incubate at 37C for 20 min before gently adding 100 μl of media dropwise.
8.4 To retrieve cells, aspirate media and add 100 μl dispase to each well.
8.5 Incubate at room temperature for 10 min, pipetting up and down occasionally.
8.6 Transfer cells and dissolved Matrigel into a 1.5 mL Eppendorf tube and wash with 1 mL 1XPBS twice.

Aspirate supernatant and either split cells in pellet or perform end point analysis.

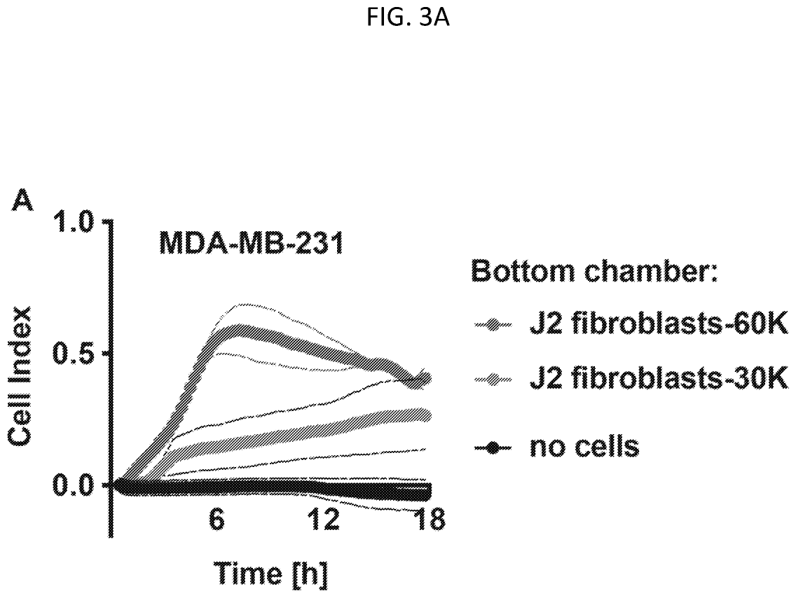

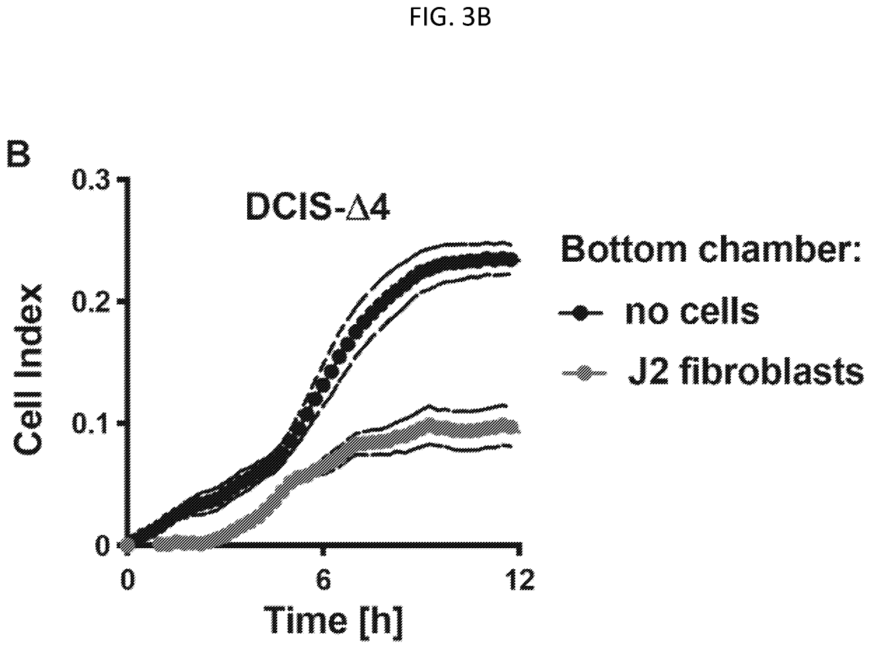

FIGS. 3A and 3B show the effect of co-cultured fibroblasts on cancer cell invasion, which is one of the types of invasive cells contemplated by the present invention. Other types of invasive cells contemplated include, but are not limited to, immune cells, epithelial cells, neuronal cells, glial cells, fibroblasts, or endothelial cells. More specifically, FIG. 3A shows Real-Time Cellular Analysis (RTCA) of MDA-MB-231 cell invasion, alone or in co-culture with 3T3-J2 fibroblasts (bottom chamber). 3T3-J2 fibroblasts were seeded at either 30,000 or 60,000 cells per well, as identified by the “30K” and “60K” designation. FIG. 3B shows Real-Time Cellular Analysis (RTCA) of DCIS-44 cell invasion, alone or in co-culture with 3T3-J2 fibroblasts (bottom chamber). The solid circles represent the mean, the thin dotted lines represent the standard deviation.

Using the newly designed three-chambered array, invasion of cells in the presence or absence of stroma cells such as fibroblasts may be detected. As shown in FIG. 3A, MDA-MB-231 cell invasion was enhanced when irradiated Swiss 3T3 fibroblasts (J2 strain) were seeded in the bottom chamber allowing for the exchange of factors between the two cell lines.

Interestingly, MDA-MB-231 invasion increased when 3T3-J2 cells were doubled in number. On the other hand, as shown in FIG. 3B, the invasion rate of an invasive clone of MCFDCIS cells (DCIS-44) appears to be inhibited by the cross talk with 3T3-J2 cells. That data shows the useful application of the three-chambered array to measure varying effects of the stroma, in this case fibroblasts, on cell invasion.

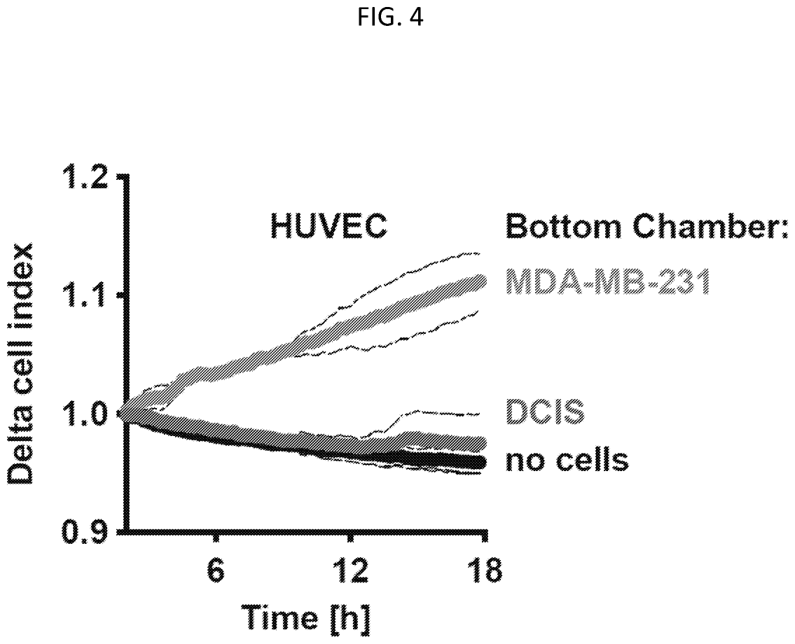

FIG. 4 shows the effect of cancer cells on endothelial cell invasion. Real-Time Cellular Analysis (RTCA) of HUVEC invasion, alone, in co-culture with the invasive MDA-MB-231 cells (bottom chamber) or the non-invasive DCIS cells (bottom chamber) is shown. The solid circles represent the mean, the thin dotted lines represent the standard deviation. The delta cell index normalized to impedance at time=1 hr.

As shown in FIG. 4, to monitor the change in endothelial cells motility and invasion in response to signals from either invasive (MDA-MB-231) or non-invasive (DCIS) cancer cells, human umbilical vein endothelial cells (HUVECs) that represent endothelial cells lining the walls of blood vessels were used. HUVECs were more invasive in response to factors secreted by MDA-MB-231 cells, unlike those secreted by DCIS cells. That is consistent with the ability of invasive tumors to recruit endothelial cells for blood vessel formation and later dissemination into the circulation. The data above demonstrate the ability of the RTCA system to monitor different invasion rates of cell lines; when invasion starts, progresses and plateaus. That allows the user to choose the time point of interest, for example, after the first 2-3 hours of invasion in order to capture the pioneer cells that initiate invasion and are distinct from the follower cells that invade thereafter by collective invasion.

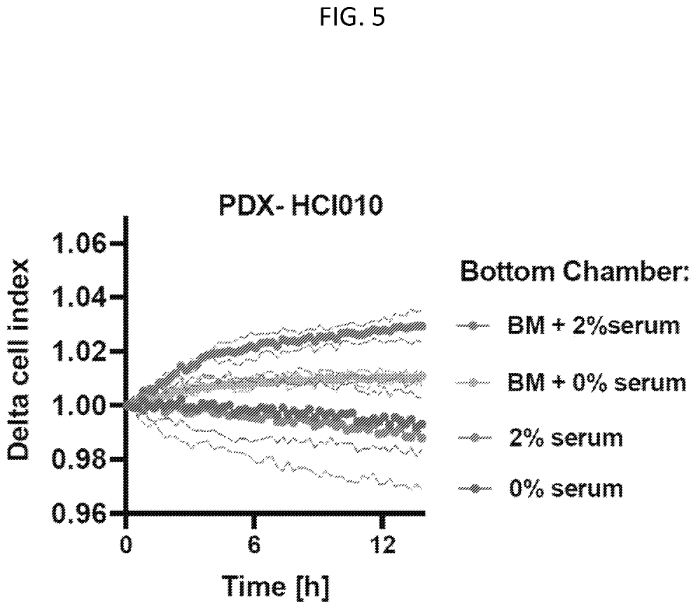

FIG. 5 shows cell invasion of patient derived xenografts (PDX) co-cultured with bone marrow immune cells. Single cells disintegrated from PDX (top chamber) were co-cultured with human bone marrow cells (bottom chamber) and their invasion monitored over time in the presence or absence of serum. The solid circles represent the mean, the thin dotted lines represent the standard deviation. The delta cell index normalized to impedance at time=1.6 hr.

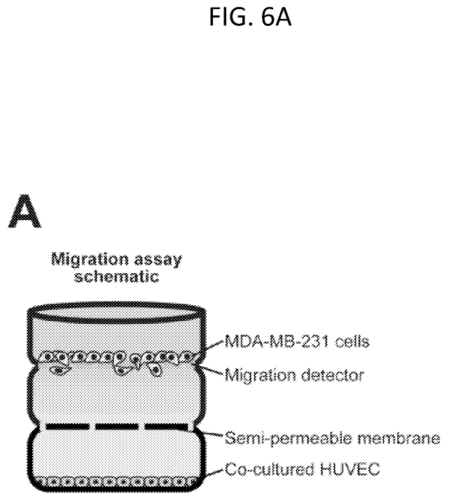

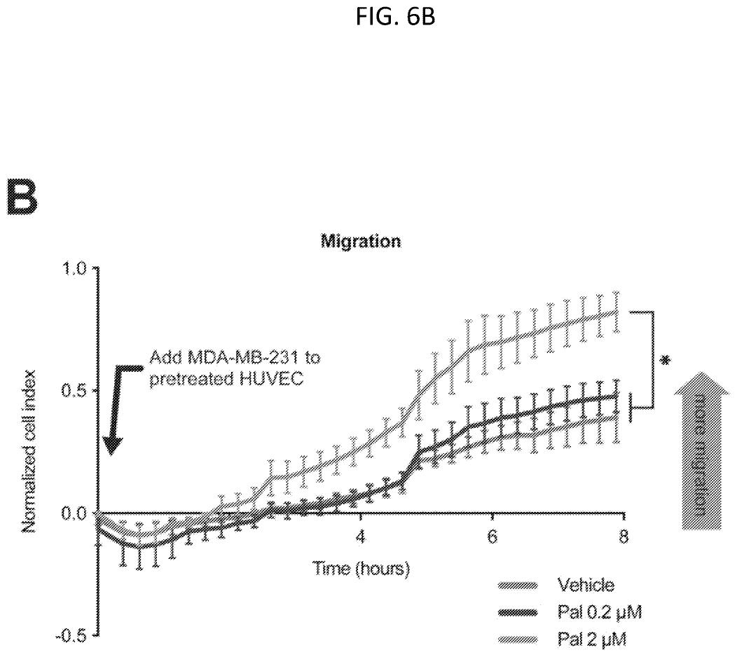

FIG. 6A is a diagram of the cell invasion monitoring and cell collection system comprising migrating MDA-MB-231 tumor cells, in which HUVEC cells in the system are treated with Palbociclib. This exemplary system was used to analyze the effects of palbociclib treatment of endothelial (HUVEC) cells on tumor cell migration. As shown in FIG. 6A, the three-chamber migration assay is comprised of a migration detector between the first and second chambers, on which MDA-MB-231 tumor cells are distributed. Below the MDA-MB-231 tumor cells is a semi-permeable membrane at the bottom of the second chamber, and below the semi-permeable membrane, in the third chamber, are co-cultured HUVEC cells.

FIG. 6B is a chart showing the migration of MDA-MB-231 cells in a system where HUVEC cells in the system are treated with palbociclib. HUVEC cells are plated on the bottom chamber and treated with a vehicle or palbociclib (Pal) at the indicated doses. MDA-MB-231 tumor cells are plated on the top chamber. Migration of the MDA-MB-231 tumor cells was monitored in real time after initiation of co-culture. As shown in the chart, there is a dose-dependent increase in cell migration, based on the amount of palbociclib administered.

While the above data demonstrates a solid proof of principle for the use of the three-chambered array to observe invasion in a co-culture setting, we wanted to test the potential usability of this array in clinical and diagnostic settings. For that we monitored the invasion of cell suspensions from patient derived xenografts (PDX) co-cultured with immune cells from human bone marrow samples. Total human bone marrow immune cells (BM) were seeded onto the bottom chamber with or without serum. PDX cells invasion from the top chamber increased in response to co-culturing with BM immune cells. Interestingly, the presence of 2% serum in the bottom chamber with the BM cells was essential for PDX invasion.

We have modified the design of a dual chambered array to include a third chamber for monitoring cell invasion in real time in the presence of stromal cells. We have observed distinct effects of co-cultured fibroblasts on invasive and non-invasive cancer cells indicating that the array can be used to distinguish between cancer cell subpopulations that respond differently to factors produced by co-cultured stromal cells. The array was also used to monitor endothelial cell invasion into stromal tissues, a critical step during blood vessels sprouting towards an angiogenic stimulus, in the presence of cancer cells of varying invasive potential. Those experiments show the versatile use of the array for cancer cell or other cell isolation.

It is recommended to optimize the number of cells to be added to each chamber. From our experience and the manufacturer's recommendation, 30-50 thousand cells are optimal. Since two cell types can be co-cultured in this array, one of which may be a primary culture of stroma cells, it is suggested to monitor the effects of different media on cells used to maintain viability. We found that starvation of cells to be monitored reduces variation between replicates. In the case that serum free growth conditions are harmful to the cells, low serum and shorter starvation times can be used. The addition of low serum amount (1-2%) may be critical for the survival of stromal cells (primary cells) in the bottom chamber, yet make sure to include the proper control conditions for data interpretation (i. e 2% serum with and without stroma cells). If invasion rates are low, more wells per experimental condition can increase the number of viable cells collected for downstream analyses. When analyzing patient biopsies, disintegration of tissue into single cells is crucial before testing on the RTCA plate. Optimizing disintegration conditions to maintain cell viability is an important step before performing the invasion analysis. It is also possible to study additional aspects on invasion such as sensitivity to drug treatment or the effect of extracellular basement matrix (ECM) components on invasion rate. ECM is an essential component of the microenvironment and has been reported to play a major role during cell invasion. Several published studies have used ECM to coat the top chamber before invasive cells are added to monitor their interaction with various ECM components.

The three chambered array is a useful tool to study co-culture interactions between invasive and stroma cells. While the proposed design guarantees a cell collection specific to the invasive cells without any stromal cells, this setup may not be optimal if the crosstalk between cells requires the physical interaction of the different cell types. Additionally, non-adherent cells that grow in suspension may not be collected in the proposed methods here (scrapping), yet a different approach in which media in the disassembled chambers containing the non-adherent cells may be collected to harvest the non-adherent cells.

While this array can be utilized for multiple areas of research that monitor cell invasion in the presence of a stromal component, here we focused on cancer cell invasion and how this approach can uncover malignant cancer cell subpopulations present in heterogeneous biological samples. The new capacity of the three chambered array used here provides a functional assay to isolate invasive subpopulations from heterogeneous cancer cell mixtures that contain more and less invasive cancer cells. Analysis of invasive and outcome-relevant cancer cell subpopulations is essential for appropriate mechanistic studies and molecular insights not obtainable or biased by the analysis of a mixed cell population. The co-culture chamber for stromal cells provides insights into cell-cell cross talk in the tumor microenvironment during progression to invasive disease.

As a step towards application to tissue samples, e.g. tumor biopsies from patients, we used cancer cells that were isolated from patient derived tumor xenografts (PDXs) and tested the impact of human bone marrow cells on invasion of tumor cells. We were able to collect the invasive cells present in the PDXs for downstream analysis i.e. RNA-seq. Assaying PDXs is the initial step towards analyzing cell subpopulations present in the heterogeneous mix of cancer cells in tumor biopsies obtained from patients. The ultimate goal will be to use such tumor biopsies and isolate subpopulations of cancer cells that invade and thus drive poor outcome due to their potential for metastatic spread. Identification of the molecular features and testing the sensitivity of these invasive subpopulations to drug treatment are future applications.

This technology can be useful in studying the subpopulations of cells with different invasive capacities. That includes (a) invasive cancer cells that invade surrounding tissues or blood and lymphatic vessels or extravasate at metastatic seeding site in distant organs (b) cells from the immune system that invade tissues to tackle pathogens or diseased cells, (c) endothelial cells that invade tissues to form new blood vessels during tissue reorganization or wound healing, as well as (d) stromal cells from the tumor microenvironment that support and invade along with cancer cells. The approach allows the inclusion of stromal crosstalk that can modulate cell motility and invasion. The feasibility studies shown here use that modified array and focuses on cancer cell invasion and the interaction with the stroma as a model system including endothelial invasion in response to differential signals from cancer cells. The approach can be extrapolated to isolate cancer cells as well as other cell types such as subpopulations of immune cells, fibroblasts or endothelial cells. We tested invasive and noninvasive established breast cancer cell lines as a proof of principle. We also used cells from patient derived xenograft (PDX) invasion in response to immune cells from human bone marrow to show feasibility for future use also in clinical diagnostic settings. PDX are patient tumor tissue that are implanted in immunocompromised or humanized mice model to allow for studying of growth, progression and treatment options for the original patient.

### REFERENCES

1. Friedl, P. & Gilmour, D. Collective cell migration in morphogenesis, regeneration and cancer. Nat Rev Mol Cell Biol 10, 445-457 (2009).
2. BOYDEN, S. The chemotactic effect of mixtures of antibody and antigen on polymorphonuclear leucocytes. J. Exp. Med. 115, 453-466 (1962).
3. Xu, Y. et al. A review of impedance measurements of whole cells. Biosens Bioelectron 77, 824-836 (2016).
4. Stylianou, D. C. et al. Effect of single-chain antibody targeting of the ligand-binding domain in the anaplastic lymphoma kinase receptor. 28, 3296-3306 (2009).
5. Abassi, Y. A., Wang, X. & Xu, X. Real time electronic cell sensing system and applications for cytotoxcity profiling and compound assays. United States Patent 1-88 (2013).
6. Abassi, Y. A. et al. Label-free, real-time monitoring of IgE-mediated mast cell activation on microelectronic cell sensor arrays. J. Immunol. Methods 292, 195-205 (2004).
7. Morton, C. L. & Houghton, P. J. Establishment of human tumor xenografts in immunodeficient mice. Nature Protocols 2, 247-250 (2007).
8. DeRose, Y. S. et al. Tumor grafts derived from women with breast cancer authentically reflect tumor pathology, growth, metastasis and disease outcomes. Nature Medicine 17, 1514-1520 (2011).
9. Sharif, G. M. et al. An AIB 1 Isoform Alters Enhancer Access and Enables Progression of Early-Stage Triple-Negative Breast Cancer. Cancer Research 81, 4230-4241 (2021).
10. R, C., M, O. & QV, C. Long-term human breast carcinoma cell lines of metastatic origin: preliminary characterization. 1978. In Vitro 14, 911-915.
11. Sharif, G. M. et al. Cell growth density modulates cancer cell vascular invasion via Hippo pathway activity and CXCR2 signaling. Oncogene 34, 5879-5889 (2015).
12. Miller, F. R., Santner, S. J., Tait, L. & Dawson, P. J. MCF10DCIS. com xenograft model of human comedo ductal carcinoma in situ. J Natl Cancer Inst 92, 1185-1186 (2000).
13. Winkler, J., Abisoye-Ogunniyan, A., Metcalf, K. J. & zenaa, W. Concepts of extracellular matrix remodelling in tumour progression and metastasis.Nature Communications 11, 1-19 (2020).
14. Nkosi, D., Sun, L., Duke, L. C. & Meckes, D. G. Epstein-Barr virus LMP 1 manipulates the content and functions of extracellular vesicles to enhance metastatic potential of recipient cells. PLOS Pathog. 16, e 1009023(2020 ).
15. Eisenberg, M. C. et al. Mechanistic modeling of the effects of myoferlin on tumor cell invasion. Proceedings of the National Academy of Sciences 108, 20078-20083 (2011).

The foregoing description and drawings should be considered as illustrative only of the principles of the invention. The invention is not intended to be limited by the preferred embodiment and may be implemented in a variety of ways that will be clear to one of ordinary skill in the art. Numerous applications of the invention will readily occur to those skilled in the art. Therefore, it is not desired to limit the invention to the specific examples disclosed or the exact construction and operation shown and described. Rather, all suitable modifications and equivalents may be resorted to, falling within the scope of the invention. All documents and patent applications cited herein are incorporated in their entireties by reference.

1. A method for identifying invasive cells comprising:
adding a first plurality of cells to a first chamber, wherein the first chamber is comprised of media, and the first plurality of cells are healthy cells, diseased cells, or a mixture of both;
adding media to a second chamber comprised of a filtration membrane;
adding a second plurality of cells and media to a third chamber comprised of media, wherein the second plurality of cells produces one or more chemoattractants or chemorepellents, said first, second, and third chambers being clipped together to form a chamber array;
mounting the chamber array to an electrical cell impedance sensing reader;
collecting impedance readouts from the electrical cell impedance sensing reader at predetermined time intervals; and
extracting invasive cells from under the filtration membrane of the second chamber, wherein the extracted invasive cells are characterized using the collected impedance readouts normalized over a predetermined time period.

2. The method of claim 1, wherein the invasive cells are immune cells, epithelial cells, neuronal cells, glial cells, fibroblasts, or endothelial cells.

3. The method of claim 1, wherein the chamber array is formed by cutting and interlocking mechanical protrusions in the first, second, and third chambers.

4. The method of claim 1, wherein the media is serum-free.

5. The method of claim 1, wherein the filtration membrane is comprised of polyethersulfone.

6. The method of claim 1, wherein the first plurality of cells is comprised of MDA-MB-231 cells.

7. The method of claim 1, wherein the second plurality of cells is comprised of human umbilical vein endothelial cells.

8. The method of claim 1, wherein a longitudinal slit is added to each side of the second chamber.

9. The method of claim 1, wherein the third chamber has sixteen wells.

10. The method of claim 1, wherein the impedance readouts comprise a calculation of impedance after equilibrium minus impedance in the absence of cells, divided by a nominal impedance value.

11. An apparatus for identifying invasive cells comprising:
a first chamber comprised of serum-free media, wherein a first plurality of cells is present in the first chamber, and wherein the first plurality of cells are healthy cells, diseased cells, or a mixture of both;
a second chamber comprised of a polyethersulfone membrane;
a third chamber comprised of media and a second plurality of cells producing one or more chemoattractants or chemorepellents, said first chamber, the second chamber, and the third chamber being clipped together to form a chamber array; and
an electrical cell impedance sensing reader to which the chamber array is mounted for collecting impedance readouts at predetermined time intervals, wherein invasive cells are extracted from under the filtration membrane of the second chamber, and wherein the extracted invasive cells are characterized using the collected impedance readouts normalized over a predetermined time period.

12. The apparatus of claim 11, wherein the invasive cells are immune cells, epithelial cells, neuronal cells, glial cells, fibroblasts, or endothelial cells.

13. The apparatus of claim 11, wherein the chamber array is formed by cutting and interlocking mechanical protrusions in the first, second, and third chambers.

14. The apparatus of claim 11, wherein the media is serum-free.

15. The apparatus of claim 11, wherein the filtration membrane is comprised of polyethersulfone.

16. The apparatus of claim 11, wherein the first plurality of cells is comprised of MDA-MB-231 cells.

17. The apparatus of claim 11, wherein the second plurality of cells is comprised of human umbilical vein endothelial cells.

18. The apparatus of claim 11, wherein a longitudinal slit is added to each side of the second chamber.

19. The apparatus of claim 11, wherein the third chamber has sixteen wells.

20. The apparatus of claim 11, wherein the impedance readouts comprise a calculation of impedance after equilibrium minus impedance in the absence of cells, divided by a nominal impedance value.
